(19) United States
(12) Patent Application Publication
(10) Pub. No.: US 2005/0267392 A1
(43) Pub. Date: Dec. 1, 2005

(54) Medical bandage product and substrate with para-aramid fiber reinforcement

(75) Inventor: John C. Evans, Charlotte, NC (US)

Correspondence Address: ADAMS EVANS P.A., 2180 TWO WACHOVIA CENTER, CHARLOTTE, NC, 28282, US

(73) Assignee: BSN Medical, Inc, Wilmington, DE (US)

(21) Appl. No.: 10/523,587

(22) PCT Filed: Feb. 4, 2004
(86) PCT No.: PCT/US04/03058

§ 371 Date: Feb. 4, 2005

(30) Foreign Application Priority Data
Oct. 23, 2003 (US) 60513873

(51) Int. Cl. A61F 13/00; A61F 5/00; A61F 15/00

(52) U.S. Cl. 602/42; 602/8; 602/58

(57) ABSTRACT

A medical bandaging product for being dispensed in predetermined lengths suitable for a given medical use, and includes an elongate sleeve formed of moisture-impervious material and sealable to prevent entry of moisture and an elongate medical material positioned in the sleeve and sealed therein against entry of moisture until use. The medical material includes a substrate formed of a single thickness layer formed of first yarns. The second yarns are integrated into the single thickness layer of the first yarns. The second yarns are high-strength, high modulus fibers for increasing the strength and dimensionally stabilizing the substrate. A reactive system is impregnated into or coated onto the substrate, the system remaining stable when maintained in substantially moisture-free conditions and hardening upon exposure to sufficient moisture to form a rigid, self supporting structure. A soft, flexible protective wrapping encloses the substrate along its length to provide a cushioning barrier between the substrate and the skin of a patient when the material is in use. A clip or other closure devices is provided for resealing the sleeve against entry of moisture after a predetermined length of the bandaging product has been dispensed for use to prevent hardening of the substrate remaining in the sleeve.

### TECHNICAL FIELD AND BACKGROUND OF THE INVENTION

This invention relates generally to the field of orthopaedic medicine, and more specifically, to the design of an improved single-layer, knitted, medical bandage incorporating a high strength, high modulus reinforcement yarn. It is an objective of the invention to advance and improve the processes, devices and materials of the orthopaedic industry. To that end, the successes of research and application of splinting materials for use in the aid of strains, sprains and fracture management have advanced the medical field and have provided great benefit to the patient.

For example, innovative improvements such as the multiple layer splinting applications have improved efficiency, and reduced time and costs in the application of a cast and/or splint system for the immobilization of a limb. Successful splinting and casting systems are intended to prevent, aid and correct deformity, relieve stress of maintaining muscle contraction and maintain skeletal alignment during medical treatment. Splinting and casting materials are invaluable to the medical profession in the application and aid to broken, sprained, strained and fractured bones. Current cast and splint materials exhibit property requisites such as strength, ease of assembly and finishing, pliability, rigidity, stretch, stiffness, elastic memory, weight and bulk of the finished splint, durability, aestetics, ease of cleaning and cost.

Materials exhibiting these requisite properties have been incorporated into BSN Medical, Inc.'s trademark ORTHOGLASS® brand splinting product, disclosed in U.S. Pat. Nos. 4,770,299, 4,899,738 and 5,003,970.

This approach is the background for the splinting and casting system of the present invention. The prior art exemplified by applicant's ORTHOGLASS® brand product discloses the use of multi-layer cast structures that have revolutionized treatment of fractures, sprains and other orthopaedic injuries by providing an acceptable product with, however, remaining issues of weight, performance and cost. These issues include substrate lamination, thickness and flexibility.

For example, many prior art splinting substrates are formed from overlaid fabric layers that are coated or impregnated with a moisture-curable resin. The splint is stored in moisture-free conditions until use to maintain flexibility, at which time a moisture-induced reaction hardens the substrate into a position whereby the splint supports and maintains an injured limb in a stationary position. The effectiveness of the substrate is determined by the design of the substrate structure. Ideally, the amount and distribution of the resin on the substrate is uniform across and through the substrate. In reality, the level of resin within the layers, while acceptable, is often inconsistent and suffers poor distribution that is apparent in thick and thin spots observed between the layers. This is particularly true when a considerable amount of time passes between manufacture and use of the splint, during which time migration and pooling of the resin towards the lower regions of the splint product slowly takes place.

The use of multi-layer substrates also inherently increases the thickness needed to provide a given strength. The laminations are often prevented from shifting and are maintained in contact with each other by sewing together of the layers, but with a disadvantageous inhibition of the desired degree of flexibility and conformability as a result.

The multi-layer substrate is also labor-intensive due to the need to alignment of the multiple substrate layers over each other prior to sewing the layers together. Variations in the accuracy of the alignment process can be exhibited in product rejected for poor quality, uneven distribution of resin, or in the presence of sharp, needle-like fibers projecting from the sides of the splint after application, necessitating removal and re-application of a new splint.

Additionally, the weight of the multi-layer design necessitated by the use of multiple layers of fabric can also affect the comfort of the wearer. While current industry standards are met in present splinting and casting systems, there yet remains the need for further improvement.

There is a need for lightweight materials capable of advancing splinting and casting systems in the areas of weight, conformability, efficiency and costs. Ideally, these advances should involve both material and processing improvements resulting in enhanced properties necessary for a successful casting and splinting product.

The prior art provides little disclosure directed specifically at lightweight materials incorporated into the substrate for a splinting and casting system. Therefore, the present invention seeks to provide a medical bandaging product incorporating a para-aramid fiber for reinforcement, strength, reduced weight, conformability, flexibility and cost reduction.

### SUMMARY OF THE INVENTION

Therefore, it is an object of the invention to provide a more efficient casting and splinting application by incorporating high strength, high modulus fibers within a single layer substrate—thereby reducing weight, providing greater conformability and rigidity, and improving product performance. The product is relatively easy to apply and is much more cost efficient than current casting and splinting applications. One such fiber is a para-aramid fiber having a number of high-performance properties such as high modulus, high specific strength and low weight. Such a combination of properties in a synthetic fiber is unusual and helps to create unique and diverse applications for the material. In the present invention, the para-aramid fiber used as reinforcement is knitted into the pillar or chain stitch of a single layer substrate. In addition to the aforementioned advances and improvements, the present invention, while exhibiting the requisite properties necessary for a successful application, ultimately seeks to provide better efficiency and quality over current casting and splinting systems.

It is another object of the invention to provide a reduction in the amount of costly fibers, such as fiberglass, utilized in the construction of current casting or splinting systems. For example, in the prior art, the substrate is constructed exclusively of fiberglass yarn with a weight range of approximately 2,000 to 3,000 grams per square meter (“gsm”), whereas the incorporation of a para-aramid fiber would net a range from approximately 1,500 to 2,000 gsm. Because the present invention is constructed of a single layer, incorporating para-aramid fibers that are light-weight and thin provides a better means for constructing an application that has a reduced weight, yet retains all the requisite properties and qualities of the former casting and splinting system with respect to strength, conformability and rigidity.

Significant disadvantages surrounding conventional splinting and casting systems include, for example, the weight imposed upon the patient once the cast or splint is applied and cured, as well as restriction of conformability and rigidity. The present invention provides a casting and splinting system impregnated or coated with a reactive system which remains stable when maintained in substantially moisture-free conditions but which hardens upon exposure to sufficient moisture to form a rigid, self-supporting structure. It is therefore an overall objective of the present invention to provide a lightweight medical bandaging product that eliminates the need to expend labor fabricating a laminated substrate, improves resin distribution and is also more flexible and dries more readily and thoroughly—preventing maceration of the skin.

It is another object of the invention to provide a medical bandaging product which can be dispensed in any desired length while preventing hardening of the remaining material until use is desired.

It is another object of the invention to provide a unitary medical bandaging product which includes a wrapping to provide a cushion against the skin of a patient.

It is another object of the invention to provide a method of constructing a medical bandaging product having the characteristics and objects described above.

These and other objects and advantages of the present invention are achieved in the preferred embodiment disclosed below by providing a medical bandaging product for being dispensed in predetermined lengths suitable for a given medical use, and comprising an elongate sleeve formed of moisture-impervious material and sealable to prevent entry of moisture and an elongate medical material positioned in the sleeve and sealed therein against entry of moisture until use. The medical material comprises a substrate formed of a single thickness layer formed of first yarns. The second yarns are integrated into the single thickness layer of the first yarns. The second yarns are comprised of high-strength, high modulus fibers for increasing the strength and dimensionally stabilizing the substrate. A reactive system is impregnated into or coated onto the substrate, the system remaining stable when maintained in substantially moisture-free conditions and hardening upon exposure to sufficient moisture to form a rigid, self supporting structure. A soft, flexible protective wrapping encloses the substrate along its length to provide a cushioning barrier between the substrate and the skin of a patient when the material is in use. Means are provided for resealing the sleeve against entry of moisture after a predetermined length of the bandaging product has been dispensed for use to prevent hardening of the substrate remaining in the sleeve.

According to one preferred embodiment of the invention, the first yarns are formed of fiberglass.

According to another preferred embodiment of the invention, the first yarns are formed of fiberglass and the second yarns are formed from fibers selected from the group consisting of para-aramid fibers, meta-aramid fibers and polybenzimidazone (PBI) fibers.

According to yet another preferred embodiment of the invention, the protective wrapping enclosing the substrate comprises a fibrous nonwoven cushion.

According to yet another preferred embodiment of the invention, the protective wrapping enclosing the substrate comprises a nonwoven polypropylene tube.

According to yet another preferred embodiment of the invention, the reactive system comprises a blended polyisocyanate, polyol, catalyst and stabilizer.

According to yet another preferred embodiment of the invention, the single layer substrate is knitted.

According to yet another preferred embodiment of the invention, the medical bandaging product is positioned within a dispensing box.

According to yet another preferred embodiment of the invention, the second yarns extend in spaced-apart relation to each other along the longitudinal axis of the substrate.

According to yet another preferred embodiment of the invention, the substrate is a double needlebar Raschel knit.

According to another preferred embodiment of the invention, a medical bandaging product is provided in roll form for being dispensed in predetermined lengths suitable for a given medical use, and comprises an elongate sleeve formed of a moisture-impervious aluminum foil laminate having an outer tear resistant plastic layer, a central aluminum foil layer and an inner heat sealable plastic layer and sealable to prevent entry of moisture, and an elongate medical material positioned in the sleeve and sealed therein against entry of moisture until use. The medical material comprises a substrate formed of a knitted single thickness layer formed of first yarns, and second yarns integrated into the single thickness layer of first yarns, the second yarns comprised of high-strength, high modulus fibers for increasing the strength and dimensionally stabilizing the substrate, the second yarns extending longitudinally along the length of the substrate in spaced-apart relation to each other. A reactive system is impregnated into or coated onto the substrate, the system remaining stable when maintained in substantially moisture-free conditions and hardening upon exposure to sufficient moisture to form a rigid, self supporting structure and comprising a blended polyisocyanate, polyol, catalyst and stabilizer. A soft, flexible protective nonwoven tubular web encloses the substrate along its length to provide a cushioning barrier between the substrate and the skin of a patient when the material is in use. Sealing means permit resealing of the sleeve against entry of moisture after a predetermined length of the bandaging product has been dispensed for use to prevent hardening of the substrate remaining in the sleeve.

According to yet another preferred embodiment of the invention, a substrate is provided for a medical bandaging product, and comprises a single thickness layer formed of first yarns, and second yarns integrated into the single thickness layer of first yarns. The second yarns are comprised of high-strength, high modulus fibers for increasing the strength and dimensionally stabilizing the substrate.

Preferably, the single layer substrate is knitted and the first yarns are formed of fiberglass.

According to yet another preferred embodiment of the invention, the first yarns are formed of fiberglass and the second yarns are formed from fibers selected from the group consisting of para-aramid fibers, meta-aramid fibers and polybenzimidazone (PBI) fibers.

According to yet another preferred embodiment of the invention, the second yarns extend in spaced-apart relation to each other along the longitudinal axis of the substrate.

According to yet another preferred embodiment of the invention, the substrate is a double needlebar Raschel knit.

According to yet another preferred embodiment of the invention, a substrate according to claim 1, wherein the reactive system is impregnated into or coated onto the substrate. The system remains stable when maintained in substantially moisture-free conditions and hardens upon exposure to sufficient moisture to form a rigid, self supporting structure. The substrate is enclosed within a soft, flexible protective wrapping along its length to provide a cushioning barrier between the substrate and the skin of a patient.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a perspective, schematic view showing the medical bandaging product being dispensed from a dispenser;

FIG. 2 is a view similar to FIG. 1, showing the unused portion of the medical bandaging product being resealed to prevent entry of moisture;

FIG. 3 is a perspective view with parts broken away of a cut length of medical material;

FIG. 4 is a vertical cross-section taken substantially along lines 4-4 of FIG. 3;

FIG. 5 is a perspective view of a length of the medical material with the substrate layer exposed for clarity; and

FIG. 6 is a perspective view of a length of the substrate according to an embodiment of the invention.

### DESCRIPTION OF THE PREFERRED EMBODIMENT AND BEST MODE

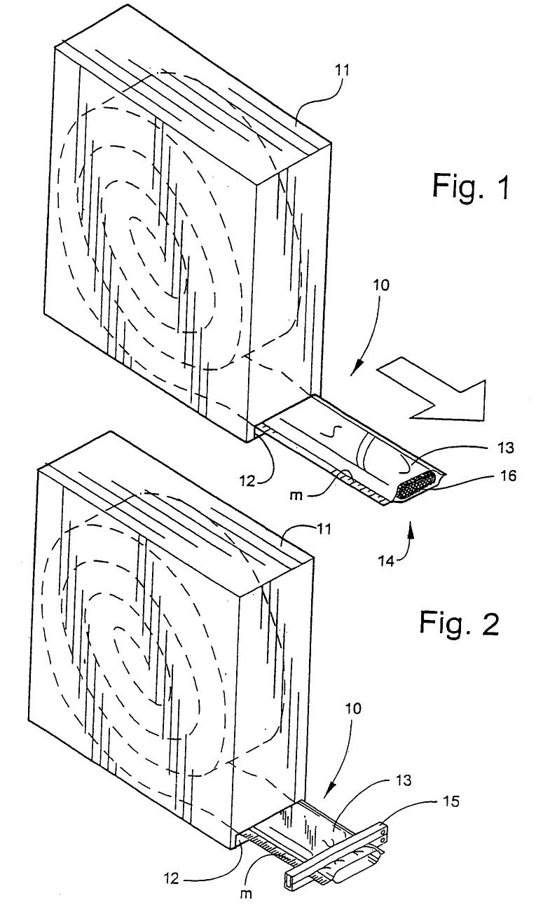

Referring now specifically to the drawings, a medical bandaging product according to the present invention is shown generally in FIG. 1 at 10. Bandaging product 10 may be sold in any convenient length, such as 24 feet, and is rolled, festooned or otherwise formed into a compact form and positioned in a suitable dispenser 11. Dispenser 11 is provided with a slot 12 at one lower corner through which bandaging product 10 extends.

Bandaging product 10 is formed generally of an outer elongate sleeve 13 which is formed of a moisture-impervious material such as a laminated plastic and foil sheet. Sleeve 13 is heat sealed along opposite, parallel extending sides to form an elongate tube. An elongate medical material 14, described in detail below, is positioned within sleeve 13 and is maintained in substantially moisture-free conditions until dispensed.

As is shown in FIG. 2, the end of sleeve 13 is sealed with sealing means, such as a clamp 15.

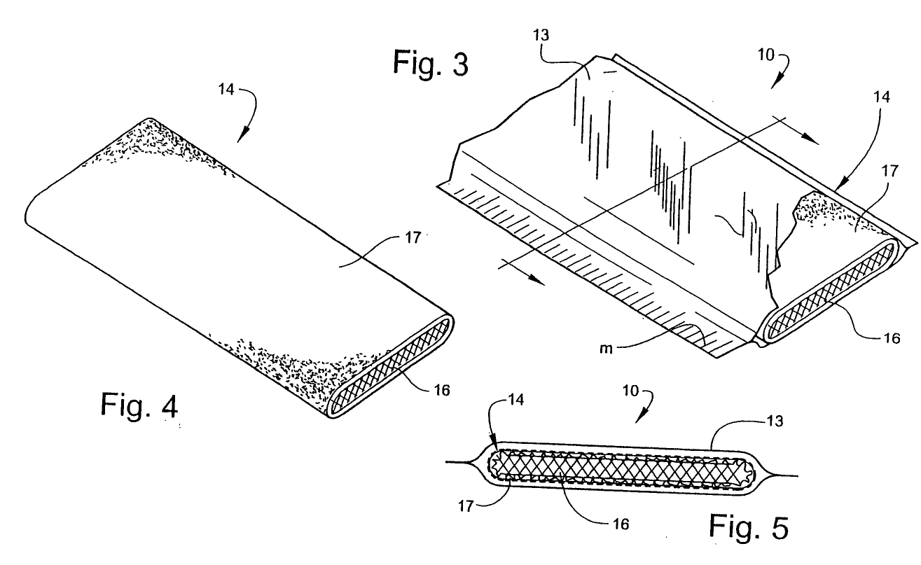

Since the appropriate length of medical material 14 is best determined by measurement, measurement marks “M” are printed on one edge of the sleeve 13, as is best shown in FIG. 3. Once the appropriate length of medical material 14 has been dispensed and cut from the roll, it is removed from sleeve 13 and sleeve 13 is discarded.

Referring now to FIGS. 4 and 5, medical material 14 is formed of a single-thickness substrate 16 constructed of knitted fiberglass yarns, with a re-enforcement of a high-strength, high modulus yarn formed of a fiber such as the para-aramid fiber disclosed more particularly below.

Substrate 16 is contained within a tubular wrapping 17 which is formed of a soft, flexible non-woven fiber such as polypropylene or some other suitable hydrophobic fiber. This provides a cushioning protective layer between the skin of the patient and substrate 16.

Substrate 16 is impregnated or coated with a reactive system which remains stable when maintained in substantially moisture-free conditions but which hardens upon exposure to sufficient moisture to form a rigid, self-supporting structure. A typical formulation of the reaction system is set forth in Table 1, below:

TABLE 1
Typical Formulation:

| | | |
|---|---|---|
| Isonate↓ 143L or | polyisocyanate | 50.0% |
| Mondur↓ CD or | | |
| Rubinate ↓ XI168 | | |
| Pluracol↓ P1010 | polyol | 46.6% |
| DC-200 Silicone | defoaming agent | 0.30% |
| Benzoyl Chloride | stabilizer | 0.10% |
| Thancat↓ DM-70 | catalyst | 3.0% |
| | | 100% |

A complete discussion of the parameters of the reactive system, the manner of production and the variables which apply are found in U.S. Pat. No. 4,411,262, referred to above.

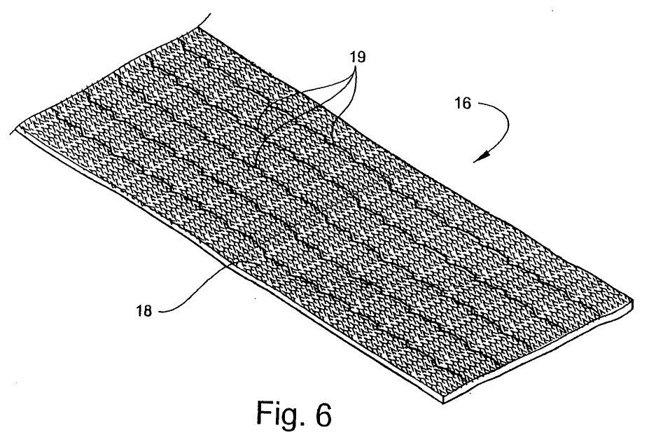

As is shown in FIG. 6, the substrate 16 is a single-layer substrate which can be woven or knitted and includes polyolefin yarns, polyester yarns and/or glass yarns 18 reinforced with high strength, high modulus yarns 19. The preferred reinforcement yarn is a yarn formed of para-aramid fibers such as Dupont's KEVLAR® or Teijin's TWARON® fibers. Other reinforcement yarns include yarns of meta-aramid or polybenzimidazole (PBI) fiber.

The preferred embodiment of the substrate is a single-layer structure knitted on a double needlebar Raschel Knitting Machine. The single-layer structure is composed of glass fiber yarns and a reinforcement yarn, Dupont's Keviar. The single-layer structure is constructed using a glass fiber textured yarn in the count range of 50 Tex-136 Tex, preferably 70 Decitex of either 6 or 9 micron glass. The para-aramid reinforcement yarn is either filament or spun with a typical count range of 10 to 100 Tex, more preferably, 70 Tex, spun yarn. The para-aramid yarn is positioned in the structure in every chain stitch on the outside bars over every needle or up to 4 needles but more preferably every 2 needles so laying the thread between 5 mm to 20 mm on the inside and outside surface but preferably every 12 mm on the inside and outside surface. The single-layer fabric is knitted within a weight range of 1500-2200 gsm but preferably 1850 gsm. The numbers of threads per centimeter in the fill direction is in a range of 20.0 to 50.0 but preferably 30.0 and in the warp or wale direction a range of 20 threads per centimeter to 50 threads per centimeter more preferably 25 threads per centimeter.

A preferred specification is set out in Table 2, below:

| | |
|---|---|
| Gauge (inch) 10 | Machine Used Barfuss |
| Courses per Meter (machine state) 320 | |

| | | Ends Per | | Feed Rate | Pattern Details | |
|---|---|---|---|---|---|---|
| Bar | Yarn | Guide | Threading | (mm/rack) | Description | Chain |
| 1 | 70 tex text glass | 2 | full | 15000 | Chain stitch on both beds, | 20/68 |
| | | | | | diagonally across the beds | |
| | | | | | Over 4 needles | |
| 2 | EGT 6 75 G1 + G1 | 2 | full | 1500 | Inlay over 4 needles in the | 88/88/00/00 |
| | Text glass (ex IGF380) | | | | center of the fabric | |
| 3 | 70 tex text glass | 2 | full | 12000 | Chain stitch on both beds, | 42/02 |
| | | | | | diagonally across the beds | |
| | | | | | Over 2 needles | |
| 4 | Spun Kevlar 19 tex | 1 | 1 in | 12000 | Chain stitch on both beds, | 42/02 |
| | | | 7 out | | diagonally across the beds | |
| | | | repeated | | Over 2 needles | |
| 5 | | | | | | |
| 6 | | | | | | |

Fabric Wight (gsm) 2200 relaxed

The para-aramid yarn dimensionally stabilizes the substrate 16 while not interfering with the flexibility and conformability of the structure.

A medical bandaging product is described above. Various details of the invention may be changed without departing from its scope. Furthermore, the foregoing description of the preferred embodiment of the invention and the best mode for practicing the invention are provided for the purpose of illustration only and not for the purpose of limitation--the invention being defined by the claims.

1. A medical bandaging product for being dispensed in predetermined lengths suitable for a given medical use, comprising:
(a) an elongate sleeve formed of moisture-impervious material and sealable to prevent entry of moisture;
(b) an elongate medical material positioned in said sleeve and sealed therein against entry of moisture until use, said medical material comprising:
(i) a substrate formed of a single thickness layer formed of first yarns, and second yarns integrated into the single thickness layer of first yarns, the second yarns comprised of high-strength, high modulus fibers for increasing the strength and dimensionally stabilizing the substrate;
(ii) a reactive system impregnated into or coated onto said substrate, said system remaining stable when maintained in substantially moisture-free conditions and hardening upon exposure to sufficient moisture to form a rigid, self supporting structure; and
(iii) a soft, flexible protective wrapping enclosing said substrate along its length to provide a cushioning barrier between the substrate and the skin of a patient when the material is in use;
(c) means for resealing said sleeve against entry of moisture after a predetermined length of said bandaging product has been dispensed for use to prevent hardening of said substrate remaining in said sleeve.

2. A medical bandaging product according to claim 1, wherein first yarns are formed of fiberglass.

3. A medical bandaging product according to claim 1, wherein the first yarns are formed of fiberglass and the second yarns are formed from fibers selected from the group consisting of para-aramid fibers, meta-aramid fibers and polybenzimidazone (PBI) fibers.

4. A medical bandaging product according to claim 1, wherein said protective wrapping enclosing the substrate comprises a fibrous nonwoven cushion.

5. A medical bandaging product according to claim 4, wherein said protective wrapping enclosing the substrate comprises a nonwoven polypropylene tube.

6. A medical bandaging product according to claim 1, wherein said reactive system comprises a blended polyisocyanate, polyol, catalyst and stabilizer.

7. A medical bandaging product according to claim 1, wherein said single layer substrate is knitted.

8. A medical bandaging product according to claim 1, wherein the medical bandaging product is positioned within a dispensing box.

9. A medical bandaging product according to claim 1, wherein the second yarns extend in spaced-apart relation to each other along the longitudinal axis of the substrate.

10. A medical bandaging product according to claim 1, wherein the substrate is a double needlebar Raschel knit.

11. A medical bandaging product in roll form for being dispensed in predetermined lengths suitable for a given medical use, comprising:
(a) an elongate sleeve formed of a moisture-impervious aluminum foil laminate having an outer tear resistant plastic layer, a central aluminum foil layer and an inner heat sealable plastic layer and sealable to prevent entry of moisture;
(b) an elongate medical material positioned in said sleeve and sealed therein against entry of moisture until use, said medical material comprising:
(i) a substrate formed of a knitted single thickness layer formed of first yarns, and second yarns integrated into the single thickness layer of first yarns, the second yarns comprised of high-strength, high modulus fibers for increasing the strength and dimensionally stabilizing the substrate, the second yarns extending longitudinally along the length of the substrate in spaced-apart relation to each other;
(ii) a reactive system impregnated into or coated onto said substrate, said system remaining stable when maintained in substantially moisture-free conditions and hardening upon exposure to sufficient moisture to form a rigid, self supporting structure and comprising a blended polyisocyanate, polyol, catalyst and stabilizer; and
(iii) a soft, flexible protective nonwoven tubular web enclosing said substrate along its length to provide a cushioning barrier between the substrate and the skin of a patient when the material is in use;
(c) means for resealing said sleeve against entry of moisture after a predetermined length of said bandaging product has been dispensed for use to prevent hardening of said substrate remaining in said sleeve.

12. A substrate for a medical bandaging product, comprising:
(a) a single thickness layer formed of first yarns; and
(b) second yarns integrated into the single thickness layer of first yarns, the second yarns comprised of high-strength, high modulus fibers for increasing the strength and dimensionally stabilizing the substrate.

13. A substrate according to claim 12, wherein said single layer substrate is knitted.

14. A substrate according to claim 12, wherein the wherein first yarns are formed of fiberglass.

15. A substrate according to claim 12, wherein the first yarns are formed of fiberglass and the second yarns are formed from fibers selected from the group consisting of para-aramid fibers, meta-aramid fibers and polybenzimidazone (PBI) fibers.

16. A substrate according to claim 12, wherein the second yarns extend in spaced-apart relation to each other along the longitudinal axis of the substrate.

17. A substrate according to claim 12, wherein the substrate is a double needlebar Raschel knit.

18. A substrate according to claim 12, wherein:
(a) the reactive system is impregnated into or coated onto the substrate, the system remaining stable when maintained in substantially moisture-free conditions and hardening upon exposure to sufficient moisture to form a rigid, self supporting structure; and
(b) the substrate is enclosed within a soft, flexible protective wrapping along its length to provide a cushioning barrier between the substrate and the skin of a patient.

19. A substrate according to claim 18, wherein the substrate is packaged within a moisture-impervious package and sealing therein until use.
